(19) United States
(12) Patent Application Publication
(10) Pub. No.: US 2005/0158380 A1
(43) Pub. Date: Jul. 21, 2005

(54) Sustained release oral dosage forms of gabapentin

(76) Inventors: Manish Chawla, Delhi (IN); Rajeev Singh Raghuvanshi, New Delhi (IN); Ashok Rampal, Amritsar (IN)

Correspondence Address: Jayadeep R Deshmukh;Ranbaxy Pharmaceutical Inc, Suite 2100, 600 College Road East, Princeton, NJ, 08540, US

(21) Appl. No.: 10/517,080

(22) PCT Filed: Jun. 6, 2003
(86) PCT No.: PCT/IB03/02166

(30) Foreign Application Priority Data
Jun. 7, 2002 (IN) 616/DEL/2002

(51) Int. Cl. A61K 31/195; A61K 9/20; A61K 9/22

(52) U.S. Cl. 424/465; 514/561; 424/468

(57) ABSTRACT

The present invention relates to sustained release oral dosage forms of gabapentin and at least one rate controlling polymer, and a process for the preparation of the sustained release oral dosage forms, and a process for the preparation thereof. The sustained release tablet includes gabapentin or a pharmaceutically acceptable salt or hydrates thereof and at least one rate-controlling polymer such that the tablet provides therapeutically effective plasma levels of gabapentin for a period of up to about 12 hours.

### FIELD OF THE INVENTION

The technical field of the invention relates to sustained release oral dosage forms of gabapentin and at least one rate controlling polymer, and a process for the preparation of the sustained release oral dosage forms.

### BACKGROUND OF THE INVENTION

Gabapentin (1-(aminomethyl)cyclohexaneacetic acid) is an γ-amino, acid analogue effective in the treatment of epilepsy. Gabapentin is indicated as an adjunctive therapy in the treatment of partial seizures with and without secondary generalization in adults with epilepsy. Gabapentin has also been approved for neuropathic pain in some countries.

Some epileptic patients need to take medication throughout their lives while others may only require it for a limited period. The importance of taking drugs at regular intervals is well known. However, it also is well known that not all patients remember to take the correct dose at the same time each day. Thus, a multiple dosing regimen is not only inconvenient but also lowers patient compliance.

Gabapentin has a relatively short half-life (i.e., 5-7 hours), which leads to substantial fluctuations in the plasma concentration of the drug. Frequent dosing is necessary to maintain reasonably stable plasma concentrations. The effective dose of gabapentin is 900 to 1800 mg/day, which is given in divided doses. Gabapentin conventional dosage forms like tablets or capsules are administered three times a day. This mode of therapy leads to sudden, high drug concentrations in the blood after dosing, followed by a rapid decrease in drug concentrations as a result of drug distribution, metabolism and elimination. The large difference in minimum and maximum plasma concentration is a major disadvantage associated with conventional dosage forms.

It has been determined that gabapentin is typically absorbed from the upper intestine, i.e., it has a narrow absorption window and is absorbed by active transport through a large neutral amino acid (LNAA) transporter. This transporter is located in the upper small intestine, has limited transport capacity, and becomes saturated at high drug concentrations. Consequently, the plasma levels of gabapentin are not dose proportional and, therefore, higher doses do not give proportionately higher plasma levels. Since the LNAA transporter responsible for gabapentin absorption is present only in the upper region of the intestine, the dosage form used to provide gabapentin should be designed to release gabapentin in the stomach at a rate such that the maximum amount of the drug is available in the intestinal segment. Conventional dosage forms release most of the gabapentin in the stomach within a short time and, consequently, there is a high likelihood that the drug is incompletely absorbed from the upper region of the intestine.

Sustained release dosage forms are designed to release drugs over an extended period of time, and usually throughout the gastrointestinal (GI) tract. Under these circumstances, drugs having a narrow absorption window tend to show poor absorption since a sustained release dosage form that includes such a drug is most likely to pass beyond the specific absorption site while still containing a substantial portion of the drug. This may result in sub-therapeutic blood levels of the drug, quick termination of drug action, and, consequently, ineffective treatment of the patient's condition.

U.S. Pat. No. 5,955,103 discloses an osmotic dosage form for sustained release of antiepileptic drugs. The dosage form includes an outer wall and an inner membrane in contact with the outer wall. Inside the dosage form, there are two layers, an expandable polymeric layer and a drug layer in contact with the expandable polymeric layer. There is an exit orifice in the outer wall and the membrane from which the drug release takes place. The outer wall maintains the integrity of the dosage form and protects the inner membrane and the enclosed layers from the variable pH environment of the gastrointestinal tract (GI). Once inside the stomach, water penetrates the dosage form and the expandable polymeric layer absorbs water and swells, thereby pushing the drug out through the orifice to the outside of the dosage form.

The process to formulate such osmotic dosage forms requires many steps of manufacturing and is expensive. There is a lag time after administration of the dosage form before drug release occurs from these dosage forms. Also, there is a likelihood of dose dumping in the event that the dosage form ruptures after contacting with food in the gastro-intestinal tract.

### SUMMARY OF THE INVENTION

In one general aspect, a sustained release tablet is provided which includes gabapentin or a pharmaceutically acceptable salt or hydrate thereof and at least one rate-controlling polymer. The tablet provides therapeutically effective plasma levels of gabapentin for a period of up to about 12 hours.

Embodiments of the sustained release tablet may include one or more of the following features or characteristics. For example, the tablet may exhibit the following in-vitro dissolution profile when measured in a USP type II dissolution apparatus at 50 rpm, a temperature of 37° C.±0.5° C. in 900 ml of 0.06 N hydrochloric acid:
at most approximately 50% of the drug is released in 1 hour,
at most approximately 65% of the drug is released in 2 hours, and
at most approximately 85% of the drug is released in 4 hours.

Administering the tablet twice per day may provide comparable bioavailability with respect to a tablet or capsule containing gabapentin administered three times per day under fasting conditions for similar cumulative daily dose. The gabapentin may be present in the tablet at from about 100 mg to about 1200 mg by weight of the tablet.

The rate-controlling polymer may be present in the tablet at from about 5% to about 80% by weight of the tablet, more particularly from about 5% to about 70% by weight of the tablet, and even more particularly at from about 5% to about 60% by weight of the tablet. The rate-controlling polymer may be one or more of polyvinylpyrrolidone, cellulosic polymer, vinylacetate copolymers, alginate, xanthan gum, guar gum, starch and starch based polymers, polyethylene oxide, methacrylic acid copolymers, maleic anhydride/methyl vinyl ether copolymers and derivatives, ethyl cellulose, cellulose acetate, methacrylates, acrylic acid polymers and copolymers, high molecular weight polyvinyl alcohols, and waxes.

The rate-controlling polymer may be a cellulosic polymer and the cellulosic polymer may be one or more of hydroxypropyl methylcellulose, hydroxypropylcellulose, hydroxyethylcellulose, and methylcellulose. The cellulosic polymer may be hydroxypropyl methylcellulose and the hydroxypropyl methylcellulose may have a viscosity of about 100 cps to about 100,000 cps, and more particularly have a viscosity of about 4,000 cps to about 15,000 cps. The cellulosic polymer may be hydroxypropylcellulose and have a viscosity of about 7 cps to about 30,000 cps, and more particularly from about 4000 cps to about 15,000 cps.

The sustained release tablet may further include one or more excipients, and the excipients may be one or more of diluent, lubricant, glidant, binder, and stabilizer. The diluent may be one or more of powdered sugar, calcium phosphate, calcium sulfate, microcrystalline cellulose, lactose, mannitol, kaolin, dry starch, and sorbitol. The lubricant may be one or more of talc, stearic acid, vegetable oil, calcium stearate, zinc stearate, and magnesium stearate. The glidant may be one or more of talc, silicon dioxide, and cornstarch. The binder may be one or more of polyvinylpyrrolidone, polyvinylpyrrolidone/vinylacetate copolymer, xanthan gum, guar gum, cellulose gums, carboxymethylcellulose, methylcellulose, hydroxypropyl methylcellulose, hydroxypropyl cellulose, gelatin, starch, and pregelatinized starch. The stabilizer may be poloxamer.

The sustained release tablet may be configured to release the gabapentin in the stomach. The tablet may release the gabapentin by a combination of diffusion and erosion. The rate controlling polymer may swell to form a polymeric matrix after contact with fluid having properties of gastric fluids.

In another general aspect, there is provided a process for the preparation of a sustained release tablet of gabapentin. The process includes granulating a mixture comprising gabapentin or a pharmaceutically acceptable salt or hydrate thereof and at least one rate-controlling polymer with one or both of water and a binder solution; and compressing the granules into a tablet. The tablet provides therapeutically effective plasma levels of gabapentin for a period of up to about 12 hours.

Embodiments of the process may include one or more of the following features or characteristics. For example, the tablet may exhibit the following in-vitro dissolution profile when measured in a USP type II dissolution apparatus at 50 rpm, a temperature of 37° C.±0.5° C. in 900 ml of 0.06 N hydrochloric acid:
at most about 50% of the drug is released in 1 hour,
at most about 65% of the drug is released in 2 hours, and
at most about 85% of the drug is released in 4 hours.

Administering the tablet twice per day may provide comparable bioavailability with respect to a tablet or capsule containing gabapentin administered three times per day under fasting conditions for similar cumulative daily dose.

The rate-controlling polymer may be present at from about 5% to about 80% by weight of the tablet and, more particularly, be present at from about 5% to about 60% by weight of the tablet. The rate-controlling polymer may be one or more of polyvinylpyrrolidone, cellulosic polymer, vinylacetate copolymers, alginate, xanthan gum, guar gum, starch and starch based polymers, polyethylene oxide, methacrylic acid copolymers, maleic anhydride/methyl vinyl ether copolymers and derivatives, ethyl cellulose, cellulose acetate, methacrylates, acrylic acid polymers and copolymers, high molecular weight polyvinyl alcohols, and waxes.

The rate-controlling polymer may be a cellulosic polymer, and cellulosic polymers may be one or more of hydroxypropyl methylcellulose, hydroxypropylcellulose, hydroxyethylcellulose, and methylcellulose. The cellulosic polymer may be hydroxypropyl methylcellulose having a viscosity of about 100 cps to about 100,000 cps and, more particularly, a viscosity of about 4,000 cps to about 15,000 cps. The cellulosic polymer may be hydroxypropylcellulose having a viscosity of about 7 cps to about 30,000 cps and, more particularly, a viscosity of about 4,000 cps to about 15,000 cps.

The mixture may further comprise one or more of diluent, lubricant, glidant, binder, and stabilizer. The diluent may be one or more of powdered sugar, calcium phosphate, calcium sulfate, microcrystalline cellulose, lactose, mannitol, kaolin, dry starch, and sorbitol. The lubricant may be one or more of talc, stearic acid, vegetable oil, calcium stearate, zinc stearate, and magnesium stearate. The glidant may be one or more of talc, silicon dioxide, and corn starch. The binder may be one or more of polyvinylpyrrolidone, polyvinylpyrrolidone/vinylacetate copolymer, xanthan gum, guar gum, cellulose gum, carboxymethylcellulose, methylcellulose, hydroxypropyl methylcellulose, hydroxypropyl cellulose, gelatin, starch, and pregelatinized starch. The stabilizer may be poloxamer.

The rate controlling polymer may swell to form a polymeric matrix after contact with fluid having properties of gastric fluids.

In another general aspect, there is provided a process for the preparation of a sustained release tablet of gabapentin. The process includes forming granules by granulating a mixture of a therapeutically effective amount of gabapentin or a pharmaceutically acceptable salt or hydrate thereof, about 5% to about 80% by weight of the tablet of hydroxypropyl methylcellulose having a viscosity of about 100 cps to about 100,000 cps, and one or more pharmaceutical excipients with water or a binder solution; and compressing the granules into a tablet. The tablet provides therapeutically effective plasma levels of gabapentin for a period of up to about 12 hours upon administration to a mammal.

In another general aspect, there is provided a process for the preparation of a sustained release tablet of gabapentin. The process includes granulating a mixture of a therapeutically effective amount of gabapentin or a pharmaceutically acceptable salt or hydrate thereof, about 5% to about 80% by weight of the tablet of hydroxypropylcellulose having a viscosity of about 7 cps to about 30,000 cps, and one or more pharmaceutical excipients with water or a binder solution; and compressing the granules into a tablet. The tablet provides therapeutically effective plasma levels of gabapentin for a period of up to about 12 hours.

In another general aspect, there is provided a method of treating a medical condition which includes providing an oral, pharmaceutical sustained release dosage form comprising gabapentin and at least one rate controlling polymer. The sustained release dosage form provides therapeutically effective plasma levels of gabapentin for a period of up to about 12 hours.

Embodiments of the method of treating may include one or more of the following. For example, the medical condition may be epilepsy. The sustained release tablet may be configured to release the gabapentin in the stomach. The sustained release tablet may release the gabapentin by a combination of diffusion and erosion. The rate controlling polymer may swell to form a polymeric matrix after contact with gastric fluids.

In another general aspect, there is provided a sustained release tablet which includes gabapentin or a pharmaceutically acceptable salt or hydrates thereof and at least one water-swellable cellulosic polymer and the tablet provides therapeutically effective plasma levels of gabapentin for a period of up to about 12 hours.

In another general aspect there is provided a sustained release tablet which includes gabapentin or a pharmaceutically acceptable salt or hydrate thereof and at least one rate-controlling polymer and the tablet has a relatively extended gastric residence time and the tablet provides for the sustained release of gabapentin in the stomach environment over a prolonged period of time.

In another general aspect there is provided a process for the preparation of a sustained release tablet which includes a mixture that includes gabapentin or a pharmaceutically acceptable salt or hydrates thereof and at least one water-swellable cellulosic polymer; compressing the granules into a tablet. The tablet provides therapeutically effective plasma levels of gabapentin for a period of up to about 12 hours.

In another general aspect there is provided a process for the preparation of a sustained release tablet, the process including granulating a mixture which includes gabapentin or a pharmaceutically acceptable salt or hydrates thereof and at least one rate-controlling polymer; compressing the granules into a tablet. The tablet has a relatively extended gastric residence time and the tablet provides for the sustained release of gabapentin in the stomach environment over a prolonged period of time.

The details of one or more embodiments of the inventions are set forth in the description below. Other features, objects, and advantages of the invention will be apparent from the description and claims.

### DETAILED DESCRIPTION OF THE INVENTION

Based on the description above, a suitably designed sustained release dosage form of gabapentin that solve the shortcomings of conventional dosage forms is desirable. Gabapentin in such a form can be designed to be given in one or two daily doses, thus requiring less frequent dosing and improving patient compliance. Effective plasma levels can be maintained within the therapeutic range with minimum of fluctuations in blood levels of gabapentin in comparison to conventional dosage forms. The steady plasma levels will reduce side effects and increase the therapeutic efficacy. Optimally, a suitably designed sustained release dosage form of gabapentin should be simple to prepare and capable of maintaining effective plasma concentration over an extended period of time would be desirable.

Based on the understanding that gabapentin has a narrow absorption window, a sustained release dosage form should be designed that can give increased exposure of gabapentin to LNAA transporter over an extended time period for efficient absorption. In order to achieve such a desirable objective, a dosage form with a sustained release mode should be designed to have a relatively extended gastric residence time in the stomach in which there can be the desired slow release of gabapentin. The controlled amount of gabapentin that is released from the dosage form will pass from the stomach to the upper intestine and become available for absorption. This will ensure that the LNAA transporter does not become saturated, thereby achieving maximal absorption of the drug.

The inventors have developed novel sustained release dosage forms, in the form of tablets, that can provide therapeutic levels of gabapentin with a reduced number of administered doses. For example, the rate and extent of absorption from the novel sustained release tablets given twice a day is the same in comparison to a conventional tablet given three times a day for similar cumulative daily dose and maintains gabapentin plasma levels in a therapeutic range over an extended period. A process for preparing the novel sustained release tablets is also described herein that is less time-consuming, can be easily carried out, and is economical.

Gabapentin is a highly water-soluble drug having a solubility of about 1 part in 20 parts of water. The desired sustained release of gabapentin, which has such a high solubility, is provided by formulating a tablet that includes dispersible gabapentin in a swellable polymeric matrix. In the presence of gastric fluids, the matrix swells by imbibing water and slowly releases the incorporated gabapentin by a combination of both diffusion and erosion. First, there is diffusion of the drug from the swollen matrix to the surrounding fluids owing to a concentration gradient of the drug between the swollen matrix and gastric fluid. Second, in the swollen state, the polymeric matrix slowly dissolves or erodes from the surface and releases the drug. Nonetheless, the tablet in its swollen state may retain its shape for sufficiently long time.

The sustained release dosage form described herein is prepared by blending gabapentin with at least one rate-controlling polymer and other excipients, wet granulating the blend with water or a binder solution, drying and sizing the wet granules, and compressing the granules into tablets. Although this process is satisfactory, other processes, including those described below, may instead be used to satisfactorily prepare the sustained release dosage form.

Gabapentin may be present as a free base, hydrated form, such as monohydrate or any other pharmaceutically acceptable salt thereof, with the anion of the mineral acid (calculated as chloride content) being less than 100 ppm and lactam content being less than 0.05% weight by weight of gabapentin, although other pharmaceutically acceptable quantities may be used. Gabapentin may comprise from about 100 mg to about 1200 mg by weight of the tablet.

The rate-controlling polymer generally may be either a hydrophilic or a hydrophobic polymer; particularly suitable are polymers that swell in aqueous media. The amount of polymer in the tablet relative to gabapentin depends upon the rate of drug release required, the type and molecular weight of the polymer, and the types of other excipients present in the formulation. Examples of suitable rate-controlling polymers include polyvinylpyrrolidone; cellulosic polymers such as hydroxypropyl methylcellulose, hydroxypropylcellulose, hydroxyethylcellulose, and methylcellulose; vinylacetate copolymers; polysaccharides, such as alginate, xanthan gum, guar gum, etc.; starch and starch based polymers; polyethylene oxide, methacrylic acid copolymers; maleic anhydride/methyl vinyl ether copolymers and derivatives and mixtures thereof.

Particularly suitable rate-controlling polymers include hydroxypropyl methylcellulose and hydroxypropylcellulose. Hydroxypropyl methylcellulose can be of various viscosity grades, such as those having a viscosity from about 100 cps to about 100,000 cps. Suitable types of HPMC are sold under the trade name Methocel by Dow Chemical Co. and include Methocel K4MCR, Methocel K15MCR, and Methocel K100MCR. Hydroxypropylcellulose also can be of various viscosity grades, and includes those sold by Aqualon under the brand name of Kluce, and those sold by Nippon Soda Co. Ltd, Japan. Suitable grades are those having viscosity of from about 7 cps to about 30,000 cps. Especially suitable among these hydroxypropylcelluloses are those having viscosity of 4,000 cps to about 30,000 cps. Besides the above, cellulose derivatives, such as ethylcellulose or cellulose acetate; methacrylates; acrylic acid polymers and copolymers; high molecular weight polyvinyl alcohols; and waxes, such as fatty acids and glycerides, are also included. The amount of polymer in the dosage form may vary from about 5% to about 80% by weight of the composition, in particular from about 5% to about 70%, and more particularly from about 5% to 60% by weight of the composition.

The sustained release gabapentin tablets as described herein may further include other additives or excipients such as diluents, lubricants, binders, stabilizers, etc. Suitable diluents include powdered sugar, calcium phosphate, calcium sulfate, microcrystalline cellulose, lactose, mannitol, kaolin, dry starch, sorbitol, etc. Suitable lubricants include talc, stearic acid, vegetable oil, calcium stearate, zinc stearate and magnesium stearate. Suitable glidants include talc, silicon dioxide, and cornstarch. Suitable binders include polyvinylpyrrolidone; polyvinylpyrrolidone/vinyl acetate copolymer, xanthan gum; guar gum; cellulose ethers, such as carboxymethylcellulose, methylcellulose, hydroxypropyl methylcellulose, and hydroxypropyl cellulose; gelatin; and starch and its derivatives. A suitable stabilizer is poloxamer, although other suitable stabilizers are contemplated.

Gabapentin sustained release tablets may be prepared according to the following steps:
1. Blend gabapentin with rate-controlling polymer(s) and optionally with other excipients in a suitable mixer.
2. Granulate the blend of step 1 with water or a binder solution.
3. Dry and size the granules.
4. Mix the sized granules with other excipients, such as one or more of diluent stabilizer, lubricant, and glidant, and compress into a tablet.

Alternatively, non-aqueous granulation, direct compression, or dry granulation techniques may be used to prepare tablets. In direct compression the blend of gabapentin, rate-controlling polymer(s), diluent, binder, stabilizer, and lubricant is prepared and compressed into a tablet. The dry granulation process can be carried out by compaction or by preparing slugs of a mixture of gabapentin, rate-controlling polymer(s) and optionally other excipients; sizing of the material/slugs so obtained; mixing with a lubricant and compressing into a tablet.

Tablets can additionally be coated with non-rate-controlling polymer(s) compositions, such as Opadry® sold by Colorcon, to impart aesthetic appeal. Such a coating may comprise up to about 2% by weight of the tablet.

The novel gabapentin sustained release tablets and the processes for the preparations described herein are further illustrated by the following illustrative examples, which are intended to exemplify but not limit the scope of the inventions.

| | Quantity (mg) | | | | | | |
|---|---|---|---|---|---|---|---|
| Ingredients | Example 1 | Example 2 | Example 3 | Example 4 | Example 5 | Example 6 | Example 7 |
| Gabapentin | 900 | 900 | 900 | 900 | 900 | 600 | 450 |
| Hydroxypropylmethyl cellulose | 100 | 250 | 100 | — | — | 100 | 75 |
| Hydroxypropylcellulose | — | — | — | 120 | 265 | | |
| Microcrystalline cellulose | 110 | — | — | — | — | | |
| Mannitol | — | 76 | 81 | 60.5 | 15.5 | 37 | 27.75 |
| Polyvinylpyrrolidone/vinyl acetate | — | 12 | 12 | 12 | 12 | 8 | 6 |
| copolymer | | | | | | | |
| Poloxamer 407 | — | 12 | 12 | 15 | 15 | 10 | 7.5 |
| Magnesium Stearate | 7.5 | 15 | 15 | 20 | 20 | 10 | 7.5 |
| Colloidal Silicon Dioxide | 7.5 | — | — | — | — | | |
| Talc | — | 15 | 15 | 22.5 | 22.5 | 10 | 7.5 |
| Total weight | 1125 | 1280 | 1135 | 1150 | 1250 | 775 | 581.25 |

Method:

### EXAMPLE 1

Gabapentin was mixed with a portion of the hydroxypropylmethylcellulose and the microcrystalline cellulose in a rapid mixer granulator and granulated with an aqueous solution of the remaining portion of hydroxypropylmethylcellulose. The wet mass was dried, suitably sized, lubricated with magnesium stearate and colloidal silicon dioxide, and compressed using appropriate tooling. The tablets were subsequently coated with OPADRY to a weight build up of about 2% w/w.

### EXAMPLES 2 AND 3

Gabapentin was mixed with mannitol and a portion of the hydroxypropylmethyl cellulose in a rapid mixer granulator and granulated with an aqueous solution/dispersion of polyvinylpyrrolidone/vinyl acetate copolymer and the remaining portion of hydroxypropyl methylcellulose. The wet mass was dried, suitably sized, mixed with poloxamer magnesium stearate, and talc, and compressed using appropriate tooling. The tablets were subsequently coated with OPADRY to a weight build up of about 2% w/w.

### EXAMPLES 4 AND 5

Gabapentin was mixed with a portion of the hydroxypropylcellulose and the mannitol in a rapid mixer granulator and granulated with an aqueous solution/dispersion of the remaining portion of hydroxypropylcellulose. The wet mass was dried, suitably sized, mixed with the remaining excipients, and compressed using appropriate tooling. The tablets were subsequently coated with OPADRY to a weight buildup of about 2% w/w.

### EXAMPLES 6 AND 7

Gabapentin was mixed with a portion of the hydroxypropylmethylcellulose and the mannitol in a rapid mixer granulator and granulated with the aqueous solution/dispersion of the remaining portion of hydroxypropylmethylcellulose. The wet mass was dried, suitably sized, blended with remaining excipients and compressed using appropriate tooling. The tablets were subsequently coated with OPADRY to a weight build up of about 2% w/w.

Tablets of Examples 1-7 were tested in dissolution studies in a USP II apparatus in 0.06N HCl (900 ml). The temperature and agitation were set at 37° C.±0.5° C. and 50 rpm, respectively. Aliquots of sample were withdrawn at predetermined time intervals and replaced with an equal amount of fresh media. Samples were processed and suitably analyzed. Dissolution profiles of these tablets are given in Table 1.

TABLE 1
Dissolution profile of tablets prepared as per the compositions of examples 1-7.

| | % Drug release | | | | | | |
|---|---|---|---|---|---|---|---|
| Time | Example | Example | Example | Example | Example | Example | Example |
| (hr) | 1 | 2 | 3 | 4 | 5 | 6 | 7 |
| 0.5 | 16 | — | 16 | 18 | — | 16 | 18 |
| 1 | 26 | 21 | 29 | 29 | 24 | 26 | 29 |
| 2 | 43 | 33 | 50 | 47 | 36 | 42 | 47 |
| 4 | 70 | 51 | 78 | 75 | 52 | 69 | 74 |
| 6 | 89 | 66 | 95 | 95 | 65 | 85 | 91 |
| 8 | 99 | 78 | 100 | 104 | 77 | 95 | 98 |
| 10 | — | 86 | — | | 87 | | |
| 12 | — | 92 | — | | 93 | | |

The tablets of Examples 1, 3, 4, 6 and 7 released almost the entire drug within 8 hours, while the tablets of Examples 2 and 5 released about 90% of drug in 12 hours. This data indicates that suitable compositions are shown for eight hour release and twelve hour release of therapeutically effective levels of gabapentin.

Bioavailability Study:

The sustained release tablets of Example 1 (Test Product) were subjected to a bioavailability study that compared the test tablet to an immediate release formulation (NEURONTIN® 600 mg) (reference product) in an open label, randomized, 2-way crossover study in 12 healthy male volunteers under fasting conditions. NEURONTIN® 600 mg was given three times per day at eight hour intervals and tablets prepared as per example 1 were given twice per day at 12 hour intervals. The Plasma Cₘₐₓ of the Reference and Test Products in individual human subjects are given in Table 2. The mean Cₘₐₓ and mean AUC₀₋₂₄ of the Test and Reference Product in the 12 human subjects are given in Table 3. The mean AUC₀₋₂₄ and Cₘₐₓ of the Test Product was comparable to that of the Reference Product at the end of 24 hours.

TABLE 2
Plasma Cₘₐₓ of Reference Product given three times per day
and the Test Product given twice per day in human subjects.

Cₘₐₓ (μg/mL)

| | Reference Product | Test Produce |
|---|---|---|
| Subject | (Neurontin ® ) | (Example 1) |
| 1 | 18.69 | 23.79 |
| 2 | 13.02 | 14.52 |
| 3 | 10.25 | 6.22 |
| 4 | 9.39 | 10.92 |
| 5 | 27.08 | 23.75 |
| 6 | 19.27 | 14.10 |
| 7 | 7.79 | 19.35 |
| 8 | 11.84 | 12.29 |
| 9 | 19.71 | 25.97 |
| 10 | 13.65 | 9.19 |
| 11 | 16.26 | 19.02 |
| 12 | 17.81 | 14.92 |

TABLE 3
Mean Cₘₐₓ and AUC₀₋₂₄ of the Test
and Reference Products in human subjects

| | Cₘₐₓ (μg/mL) | AUC₀₋₂₄ (μg · h/mL) |
|---|---|---|
| Test Product (Example 1) | 16.17 | 199.32 |
| Reference Product | 15.40 | 199.57 |
| (Neurontin ® 600 mg) | | |

While several particular forms of the invention have been described, it will be apparent that various modifications and combinations of the invention detailed in the text can be made without departing from the spirit and scope of the invention. Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. A sustained release tablet comprising:
gabapentin or a pharmaceutically acceptable salt or hydrate thereof; and
at least one rate-controlling polymer;
wherein the tablet provides therapeutically effective plasma levels of gabapentin for a period of up to about 12 hours.

2. The sustained release tablet of claim 1, wherein the tablet exhibits the following in-vitro dissolution profile when measured in a USP type II dissolution apparatus at 50 rpm, a temperature of 37° C.±0.5° C. in 900 ml of 0.06 N hydrochloric acid:
at most approximately 50% of the drug is released in 1 hour,
at most approximately 65% of the drug is released in 2 hours, and
at most approximately 85% of the drug is released in 4 hours.

3. The sustained release tablet of claim 1, wherein administering the tablet twice per day provides comparable bioavailability with respect to a tablet or capsule containing gabapentin administered three times per day under fasting conditions for similar cumulative daily dose.

4. The sustained release tablet of claim 1, wherein the gabapentin comprises from about 100 mg to about 1200 mg by weight of the tablet.

5. The sustained release tablet of claim 1, wherein the rate-controlling polymer comprises from about 5% to about 80% by weight of the tablet.

6. The sustained release tablet of claim 5, wherein the rate-controlling polymer comprises from about 5% to about 70% by weight of the tablet.

7. The sustained release tablet of claim 6, wherein the rate-controlling polymer comprises from about 5% to about 60% by weight of the tablet.

8. The sustained release tablet of claim 1, wherein the rate-controlling polymer comprises one or more of polyvinylpyrrolidone, cellulosic polymer, vinylacetate copolymers, alginate, xanthan gum, guar gum, starch and starch based polymers, polyethylene oxide, methacrylic acid copolymers, maleic anhydride/methyl vinyl ether copolymers and derivatives, ethyl cellulose, cellulose acetate, methacrylates, acrylic acid polymers and copolymers, high molecular weight polyvinyl alcohols, and waxes.

9. The sustained release tablet of claim 8, wherein the rate-controlling polymer comprises a cellulosic polymer.

10. The sustained release tablet of claim 9, wherein the cellulosic polymer comprises one or more of hydroxypropyl methylcellulose, hydroxypropylcellulose, hydroxyethylcellulose, and methylcellulose.

11. The sustained release tablet of claim 10, wherein the cellulosic polymer comprises hydroxypropyl methylcellulose.

12. The sustained release tablet of claim 11, wherein the hydroxypropyl methylcellulose has a viscosity of about 100 cps to about 100,000 cps.

13. The sustained release tablet of claim 12, wherein the hydroxypropyl methylcellulose has a viscosity of about 4,000 cps to about 15,000 cps.

14. The sustained release tablet of claim 10, wherein the cellulosic polymer comprises hydroxypropylcellulose.

15. The sustained release tablet of claim 14, wherein the hydroxypropylcellulose has a viscosity of about 7 cps to about 30,000 cps.

16. The sustained release tablet of claim 15, wherein the hydroxypropylcellulose has a viscosity of about 4000 cps to about 15,000 cps.

17. The sustained release tablet of claim 10, wherein the cellulosic polymer comprises hydroxyethylcellulose.

18. The sustained release tablet of claim 1, further comprising one or more excipients, wherein the excipients comprise one or more of diluents, lubricants, glidants, binders, and stabilizers.

19. The sustained release tablet of claim 18, wherein the diluent comprises one or more of powdered sugar, calcium phosphate, calcium sulfate, microcrystalline cellulose, lactose, mannitol, kaolin, dry starch, and sorbitol.

20. The sustained release tablet of claim 18, wherein the lubricant comprises one or more of talc, stearic acid, vegetable oil, calcium stearate, zinc stearate, and magnesium stearate.

21. The sustained release tablet of claim 18, wherein the glidant comprises one or more of talc, silicon dioxide, and cornstarch.

22. The sustained release tablet of claim 18, wherein the binder comprises one or more of polyvinylpyrrolidone, polyvinylpyrrolidone/vinylacetate copolymer, xanthan gum, guar gum, cellulose gums, carboxymethylcellulose, methylcellulose, hydroxypropyl methylcellulose, hydroxypropyl cellulose, gelatin, starch, and pregelatinized starch.

23. The sustained release tablet of claim 18, wherein the stabilizer comprises poloxamer.

24. The sustained release tablet of claim 1, wherein the tablet is configured to release the gabapentin in the stomach.

25. The sustained release tablet of claim 1, wherein the tablet releases the gabapentin by a combination of diffusion and erosion.

26. The sustained release tablet of claim 1, wherein the rate controlling polymer swells to form a polymeric matrix after contact with fluid having properties of gastric fluids.

27. A process for the preparation of a sustained release tablet of gabapentin, the process comprising:
granulating a mixture comprising gabapentin or a pharmaceutically acceptable salt or hydrate thereof and at least one rate-controlling polymer with one or both of water and a binder solution; and
compressing the granules into a tablet,
wherein the tablet provides therapeutically effective plasma levels of gabapentin for a period of up to about 12 hours.

28. The process of claim 27, wherein the tablet exhibits the following in-vitro dissolution profile when measured in a USP type II dissolution apparatus, at 50 rpm, a temperature of 37° C.±0.5° C. in 900 ml of 0.06 N hydrochloric acid:
at most about 50% of the drug is released in 1 hour,
at most about 65% of the drug is released in 2 hours, and
at most about 85% of the drug is released in 4 hours.

29. The process of claim 27, wherein administering the tablet twice per day provides comparable bioavailability with respect to a tablet or capsule containing gabapentin administered three times per day under fasting conditions for similar cumulative daily dose.

30. The process of claim 27, wherein the rate-controlling polymer comprises from about 5% to about 80% by weight of the tablet.

31. The process of claim 27, wherein the rate-controlling polymer comprises from about 5% to about 60% by weight of the tablet.

32. The process of claim 27, wherein the rate-controlling polymer comprises one or more of polyvinylpyrrolidone, cellulosic polymer, vinylacetate copolymers, alginate, xanthan gum, guar gum, starch and starch based polymers, polyethylene oxide, methacrylic acid copolymers, maleic anhydride/methyl vinyl ether copolymers and derivatives, ethyl cellulose, cellulose acetate, methacrylates, acrylic acid polymers and copolymers, high molecular weight polyvinyl alcohols, and waxes.

33. The process of claim 32, wherein the rate-controlling polymer comprises a cellulosic polymer.

34. The process of claim 33, wherein the cellulosic polymer comprises one or more of hydroxypropyl methylcellulose, hydroxypropylcellulose, hydroxyethylcellulose, and methylcellulose.

35. The process of claim 34, wherein the cellulosic polymer comprises hydroxypropyl methylcellulose having a viscosity of about 100 cps to about 100,000 cps.

36. The process of claim 34, wherein hydroxypropyl methylcellulose has a viscosity of about 4,000 cps to about 15,000 cps.

37. The process of claim 34, wherein the cellulosic polymer comprises hydroxypropylcellulose having a viscosity of about 7 cps to about 30,000 cps.

38. The process of claim 37, wherein the hydroxypropylcellulose has a viscosity of about 4,000 cps to about 15,000 cps.

39. The process of claim 34, wherein the cellulosic polymer comprises hydroxyethylcellulose.

40. The process of claim 27, wherein the mixture further comprises one or more of diluent, lubricant, glidant, binder, and stabilizer.

41. The process of claim 40, wherein the diluent comprises one or more of powdered sugar, calcium phosphate, calcium sulfate, microcrystalline cellulose, lactose, mannitol, kaolin, dry starch, and sorbitol.

42. The process of claim 40, wherein the lubricant comprises one or more of talc, stearic acid, vegetable oil, calcium stearate, zinc stearate, and magnesium stearate.

43. The process of claim 40, wherein the glidant comprises one or more of talc, silicon dioxide, and cornstarch.

44. The process of claim 40, wherein the binder comprises one or more of polyvinylpyrrolidone, polyvinylpyrrolidone/vinylacetate copolymer, xanthan gum, guar gum, cellulose gum, carboxymethylcellulose, methylcellulose, hydroxypropyl methylcellulose, hydroxypropyl cellulose, gelatin, starch, and pregelatinized starch.

45. The process of claim 40, wherein the stabilizer comprises poloxamer.

46. The sustained release tablet of claim 27, wherein the rate controlling polymer swells to form a polymeric matrix after contact with fluid having properties of gastric fluids.

47. A process for the preparation of a sustained release tablet of gabapentin, the process comprising:
forming granules by granulating a mixture of a therapeutically effective amount of gabapentin or a pharmaceutically acceptable salt or hydrate thereof, about 5% to about 80% by weight of the tablet of hydroxypropyl methylcellulose having a viscosity of about 100 cps to about 100,000 cps, and one or more pharmaceutical excipients with water or a binder solution; and
compressing the granules into a tablet,
wherein the tablet provides therapeutically effective plasma levels of gabapentin for a period of up to about 12 hours upon administration to a mammal.

48. A process for the preparation of sustained release tablet of gabapentin, the process comprising:
granulating a mixture of a therapeutically effective amount of gabapentin or a pharmaceutically acceptable salt or hydrate thereof, about 5% to about 80% by weight of the tablet of hydroxypropylcellulose having a viscosity of about 7 cps to about 30,000 cps, and one or more pharmaceutical excipients with water or a binder solution; and compressing the granules into a tablet;
wherein the tablet provides therapeutically effective plasma levels of gabapentin for a period of up to about 12 hours.

49. A method of treating a medical condition, the method comprising providing an oral, pharmaceutical sustained release dosage form comprising gabapentin and at least one rate controlling polymer,
wherein the sustained release dosage form provides therapeutically effective plasma levels of gabapentin for a period of up to about 12 hours.

50. The method of treatment according to claim 49, wherein the medical condition comprises epilepsy.

51. The method of treatment of claim 49, wherein the sustained release tablet is configured to release the gabapentin in the stomach.

52. The method of treatment of claim 49, wherein the sustained release tablet releases the gabapentin by a combination of diffusion and erosion.

53. The method of treatment of claim 49, wherein the rate controlling polymer swells to form a polymeric matrix after contact with gastric fluids.
